(19) United States
(12) Patent Application Publication
(10) Pub. No.: US 2011/0144562 A1
(43) Pub. Date: Jun. 16, 2011

(54) Localized Pharmacological Treatment of Ocular Tissue Using High-Intensity Pulsed Electrical Fields

(75) Inventors: Tammo Heeren, Aliso Viejo, CA (US); John C. Huculak, Mission Viejo, CA (US); Steven W. Kovalcheck, Aliso Viejo, CA (US)

(73) Assignee: Alcon Research, LTD., Fort Worth, TX (US)

(21) Appl. No.: 12/637,302

(22) Filed: Dec. 14, 2009

(51) Int. Cl. A61F 9/00; A61K 9/127; A61P 27/02; A61N 1/30

(52) U.S. Cl. 604/20; 424/450

(57) ABSTRACT

A high-intensity pulsed-electrical-field (HIPEF) apparatus treats ocular tissue within a localized portion of an eye with a pharmacological solution. To mitigate risk of damage to adjacent, healthy ocular tissue, the apparatus delivers the solution to only a portion of the eye and then alters the effectiveness of at least some of the solution delivered by applying a HIPEF. In some embodiments, for example, the apparatus delivers an inactive pharmacological solution and then activates at least some of the solution by applying a HIPEF to that solution. As the apparatus applies the HIPEF with high precision, the HIPEF only activates solution within select portions of the eye. In other embodiments, the apparatus delivers a pharmacological carrier encapsulating an active pharmacological solution and then penetrates the carrier by applying a HIPEF. Delivered with a high concentration, but low dose, the solution diffuses only to tissue within a localized portion of the eye.

### TECHNICAL FIELD

The present invention relates generally to the field of eye surgery and more particularly to methods and apparatus for localized pharmacological treatment of ocular tissue during eye surgery using high-intensity pulsed electric fields.

### BACKGROUND

Pharmacological treatment of ocular tissue frequently accompanies eye surgery, e.g., to treat retinal disorders, optical nerve disorders, and the like. For example, anti-vascular endothelial growth factor agents, neuroprotectants, antioxidants, corticosteroids, and other pharmacological solutions may be applied during eye surgery to ocular tissue suffering from such disorders.

Pharmacological solutions such as these, however, often do not just affect ocular tissue intended for treatment. Rather, they tend to also affect any adjacent, healthy ocular tissue, since diffusion of the solution and other practicalities complicate precise control of which tissues are treated. As exposure of healthy ocular tissue to these pharmacological solutions may result in unacceptable tissue damage or harmful side effects, reliable approaches for localizing pharmacological treatment of ocular tissue would significantly contribute to the success of eye surgery.

### SUMMARY

As described more fully below, embodiments of the present invention treat ocular tissue within a localized portion of an eye during eye surgery with a pharmacological solution. To mitigate risk of damage to adjacent, healthy ocular tissue, the present invention delivers the pharmacological solution to only a portion of the eye and then alters the effectiveness of at least some of the solution delivered by applying a high-intensity pulsed electrical field (HIPEF) to that solution, using a HIPEF probe.

More particularly, a high-intensity pulsed electrical field (HIPEF) apparatus includes a HIPEF probe, an irrigation system, and a high voltage generator. The irrigation system delivers a pharmacological solution to ocular tissue within a portion of the eye, via an irrigation channel in the HIPEF probe or a cannula independent from the HIPEF probe. The HIPEF probe then alters the effectiveness of at least some of the solution delivered, by applying a HIPEF generated by the high voltage pulse generator.

In some embodiments, for example, the irrigation system delivers an inactive pharmacological solution that does not substantially affect treatment of ocular tissue, but the HIPEF probe activates the solution by applying the HIPEF. As the HIPEF probe applies the HIPEF with high precision, the HIPEF activates the solution, and thereby renders the solution effective for treating ocular tissue, only within select and localized portions of the eye.

In other embodiments, the irrigation system delivers a pharmacological carrier encapsulating an active pharmacological solution. Although the solution is active, the solution has no effect on treatment of ocular tissue because it is encapsulated within the carrier. The HIPEF probe then penetrates the pharmacological carrier by applying a HIPEF to the carrier, thereby exposing the tissue to the active solution and rendering the solution effective in treating that tissue. The solution is delivered in a way, e.g., with a high concentration and low dose, such that it diffuses only to ocular tissue within a localized portion of the eye and not to adjacent, healthy ocular tissue.

With the above described advantages, the present invention is particularly well suited in the context of treating retinal tissue suffering from various retinal disorders. For example, the present invention may selectively treat retinal tissue within a localized portion of the eye, without significantly affecting adjacent, healthy retinal tissue.

Of course, those skilled in the art will appreciate that the present invention is not limited to the above features, advantages, contexts or examples, and will recognize additional features and advantages upon reading the following detailed description and upon viewing the accompanying drawings.

### BRIEF DESCRIPTION OF THE DRAWINGS

FIG. 1 is a perspective view of an exemplary high-intensity pulsed electric field (HIPEF) probe used for intraocular posterior surgery.

FIG. 2 is an enlarged perspective view of the tip of the probe of FIG. 1.

FIG. 3 is a schematic diagram of a high-intensity pulsed electric field (HIPEF) apparatus according to some embodiments of the invention.

FIGS. 4A-4C illustrate various embodiments for treating ocular tissue within a localized portion of an eye with a pharmacological solution, by altering the effectiveness of the solution with a high-intensity pulsed electric field.

FIG. 5 is a logic flow diagram illustrating one embodiment of a method for treating ocular tissue within a localized portion of an eye with a pharmacological solution.

### DETAILED DESCRIPTION

The present disclosure describes an apparatus and method for treating ocular tissue within a localized portion of an eye during eye surgery with a pharmacological solution. By localizing pharmacological treatment of ocular tissue, the apparatus and method mitigate the risk of damage to adjacent, healthy tissue.

In one embodiment, the apparatus and method treat ocular tissue with a pharmacological solution using a high-intensity pulsed-electrical-filed (HIPEF) apparatus similar to that described by Steven W. Kovalceck in U.S. patent application Ser. No. 11/608,877, filed 11 Dec. 2006 and titled “System For Dissociation and Removal of Proteinaceous Tissue” (hereinafter “the Kovalcheck application”), the entire contents of which are incorporated herein by reference.

The Kovalcheck application describes using a high-intensity pulsed-electrical-field (HIPEF) rather than classical mechanical means historically used to engage, decompose, and remove vitreous tissue. The application of such a rapidly changing electrical field causes a local temporary dissociation of the adhesive and structural relations in components of vitreous tissue, thereby enabling vitreous tissue to be detached from the retinal membrane and removed from the vitreous cavity.

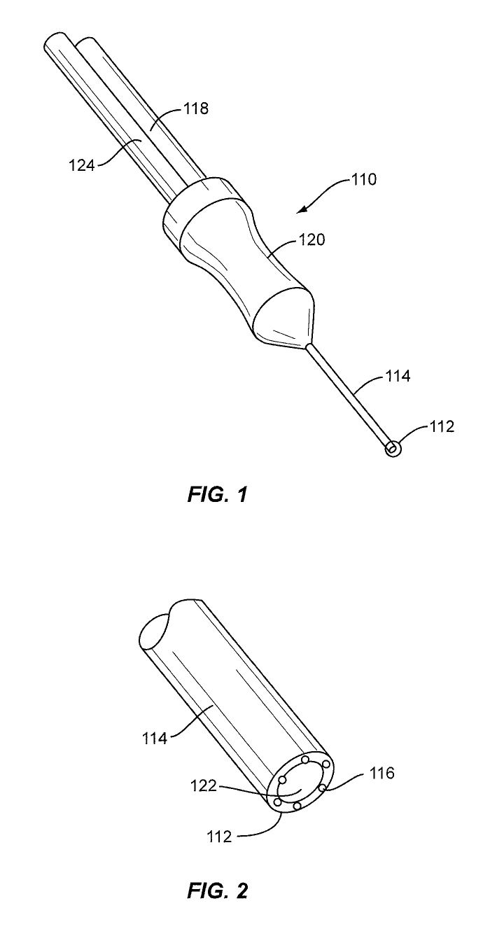

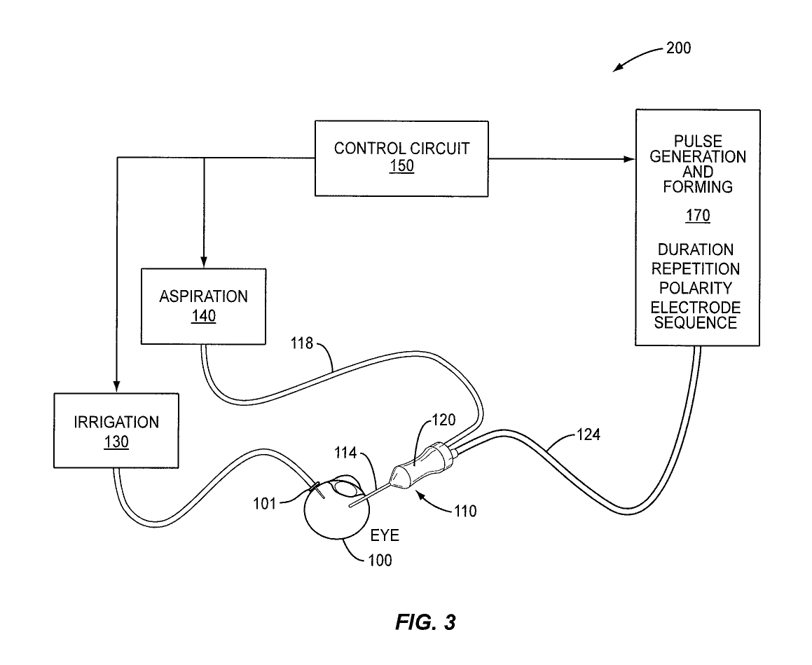

More particularly, the HIPEF is applied to vitreous tissue using a HIPEF probe 110 shown in FIG. 1. The HIPEF probe 110 comprises a hollow probe needle 114 extending from a handle 120 to a probe needle tip 112, as well as an aspiration line 118 and a transmission line 124. FIG. 2 illustrates details of the probe needle 114 and probe needle tip 112. As shown in FIG. 2, a plurality of electrodes 116 are exposed at the tip 112 and surround an aspiration lumen 122. The plurality of electrodes 116 are connected to the transmission line 124 for applying the HIPEF and disassociating vitreous tissue. The aspiration lumen 122 is connected to the aspiration line 118 for providing an aspiration pathway for disassociated vitreous tissue.

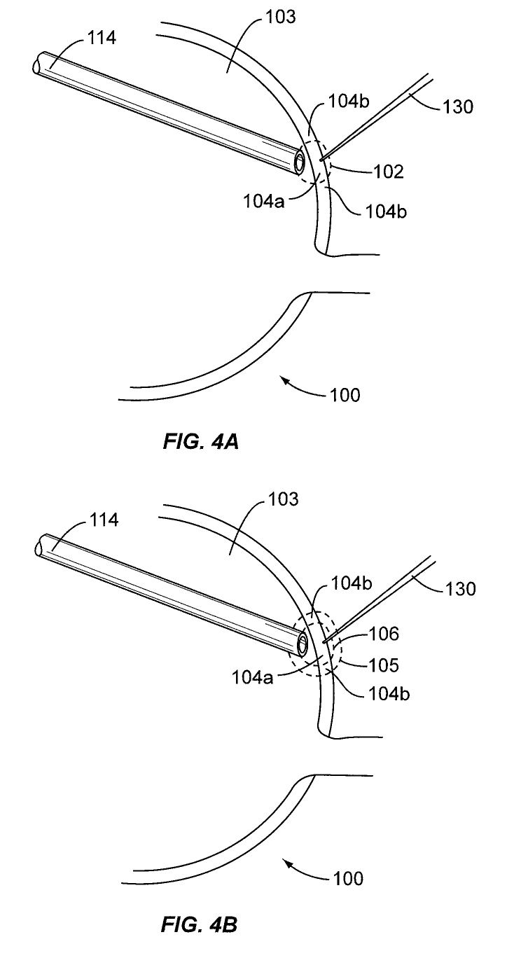

FIG. 3 illustrates additional operational details for applying a HIPEF to vitreous tissue with a HIPEF apparatus 200, which includes the HIPEF probe 110. Using handle 120, the tip 112 of the probe 110 may be inserted by a surgeon into the posterior region of an eye 100 via a pars plana approach 101. Using standard visualization processes, vitreous tissue is engaged by the tip 112 at the distal end of the hollow probe 114. An irrigation system 130 and an aspiration system 140 of the apparatus 200 are activated, by control circuit 150. The vitreous tissue is drawn into the orifice of the aspiration lumen 122 by the aspiration system 140, and then mixed with irrigation fluid delivered by the irrigation system 130 to control the electrical impedance of the vitreous tissue. Meanwhile, a high voltage pulse generator 170 of the apparatus 200 (which includes a pulse-forming network and switching circuit, in some embodiments) generates ultra-short high-intensity pulsed electric energy and sends that energy to the tip 112 via the transmission line 124 and electrodes 116. As the vitreous tissue traverses the high-intensity ultra-short-pulsed directionally changing electric field (HIPEF) concentrated at the tip 112, the adhesive mechanisms of the entrained volume of vitreous tissue are dissociated. The dissociated vitreous tissue is then removed from the vitreous cavity and drawn through the aspiration line 118 by the aspiration system 140 e.g., to a collection module.

To treat ocular tissue for a disorder, e.g., after removing vitreous tissue, the HIPEF apparatus 200 as disclosed herein is further configured to deliver irrigation fluid that consists wholly or partly of a pharmacological solution. In one embodiment, for example, the HIPEF apparatus 200 delivers a pharmacological solution that treats retinal tissue suffering from a specific retinal disorder. However, the pharmacological solution delivered also affects healthy tissue adjacent the tissue intended for treatment. Accordingly, to selectively treat ocular tissue within a localized area, without also affecting adjacent tissue, the HIPEF apparatus 200 delivers the pharmacological solution to ocular tissue only within a portion of the eye 100 and then alters the effectiveness of at least some of the pharmacological solution on treatment of ocular tissue by applying a HIPEF to that solution.

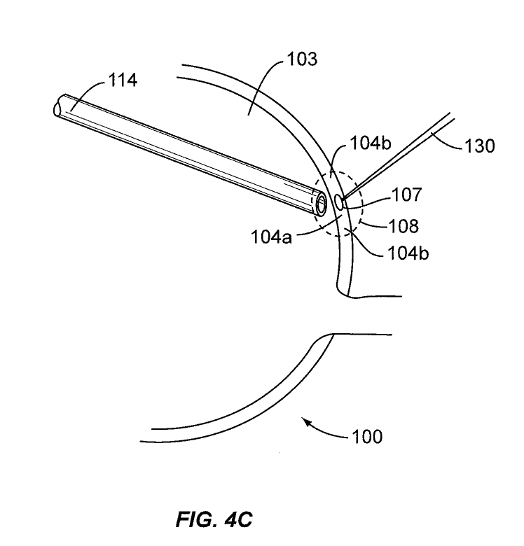

In the embodiment illustrated in FIG. 4A, for example, the HIPEF probe 114 has removed vitreous tissue from the vitreous cavity 103 as described above. The irrigation system 130 is configured to then deliver an inactive pharmacological solution to retinal tissue 104a within a portion 102 of the eye 100 suffering from a retinal disorder. The irrigation system 130, for instance, may deliver a high concentration of the inactive pharmacological solution directly to that retinal tissue 104a, but in a low dose so that its concentration rapidly decreases spatially and thereby remains substantially localized to the portion 102. Of course, as the pharmacological solution is inactive, it has no substantial effect on treatment of the retinal tissue 104a. Yet the HIPEF probe 110 in this embodiment is configured to activate the inactive pharmacological solution by applying a HIPEF to that solution. Now activated, the pharmacological solution is effective in treating retinal tissue 104a within the portion 102 as intended. Moreover, since the now activated pharmacological solution is localized to retinal tissue 104a within the portion 102, the solution does not substantially affect adjacent and healthy retinal tissue 104b.

Note that the HIPEF probe 110 primarily enables localized treatment of ocular tissue because of the highly precise and localized HIPEF, not necessarily the highly localized delivery of the pharmacological solution. That is, perfectly localized delivery of the solution to only intended ocular tissue may be substantially unattainable, e.g., even if the solution is delivered in a high concentration and low dose, the solution may nonetheless incidentally diffuse to adjacent, healthy ocular tissue. Notwithstanding this imprecise delivery, the HIPEF probe 110 is configured to apply the HIPEF with high precision, so as to only activate pharmacological solution within select and localized portions of the eye 100 that contain the intended ocular tissue.

Take, for instance, the example in FIG. 4B. In FIG. 4B, the irrigation system 130 delivers the inactive pharmacological solution to ocular tissue within a portion 105 that is larger than the portion 102 shown in FIG. 4A, such that some of the solution is incidentally delivered to healthy retinal tissue 104b. Though delivered to healthy retinal tissue 104b, the inactive solution may not substantially affect that tissue 104b without being activated. Accordingly, the HIPEF probe 110 applies a highly precise and localized HIPEF to activate only some of the pharmacological solution delivered within the portion 105, namely the solution delivered to retinal tissue 104a within the portion 106 intended for treatment.

In the above embodiments, the HIPEF apparatus 200 has been configured to deliver an inactive pharmacological solution to ocular tissue and then alter that solution's effectiveness on treatment of ocular tissue by activating the solution with a HIPEF. Those skilled in the art will appreciate, however, that the present invention is not limited to these embodiments. Indeed, other embodiments are described below with reference to FIG. 4C.

In FIG. 4C, the irrigation system 130 is configured to deliver a pharmacological carrier 107 encapsulating an active pharmacological solution to retinal tissue 104a within a portion 108 of the eye 100 intended for treatment with the solution. The pharmacological carrier 107 may be, for instance, a liposome carrier, coating, or other structure for surrounding the solution. Regardless of the specific type, the carrier 107 encapsulates the active pharmacological solution and thereby prevents the solution from being absorbed into the retinal tissue 104a. Thus, although the pharmacological solution is active, the solution has no substantial effect on treatment of the retinal tissue 104a because the solution is not absorbed into that tissue 104a. Yet the HIPEF probe 110 is configured to penetrate the pharmacological carrier 107 by applying a HIPEF to the carrier 107. In penetrating the carrier 107, the HIPEF probe 110 increases the permeability of the carrier, thereby exposing the retinal tissue 104a to the active pharmacological solution previously encapsulated by the carrier 107. As the active pharmacological is absorbed into the retinal tissue 104a, the solution becomes effective in treating that tissue 104a. Notably, the irrigation system 130 is configured to deliver a carrier 107 that encapsulates e.g., a high concentration, but low dose, of the pharmacological solution, so that the solution once exposed only diffuses to retinal tissue 104a within portion 108 and not to healthy retinal tissue 104b.

Regardless of whether the HIPEF probe 110 is configured to alter the effectiveness of the pharmacological solution by activating the solution or by penetrating a carrier of the solution, the HIPEF probe 110 generates the pulse shape, the pulse repetition rate, the pulse train length, and other parameters of the HIPEF based on the chemical properties of the solution and/or carrier. The parameters of a HIPEF for altering the effectiveness of a specific pharmacological solution may be, for instance, pre-configured in the HIPEF apparatus 200 for that solution. The parameters of a HIPEF for altering the effectiveness of one or more different pharmacological solutions may also be pre-configured in the HIPEF apparatus 200, whereby a surgeon may select between different pre-configurations based on the pharmacological solution being delivered.

Moreover, FIGS. 1-4 above have illustrated the irrigation system 130 as being configured to deliver the pharmacological solution by way of cannula independent from the HIPEF probe 110. However, those of ordinary skill in the art will understand that the irrigation system 130 may additionally or alternatively be configured to deliver the solution through one or more irrigation channels within the probe 110.

Furthermore, although the approach taught herein has been described above in the context of selectively treating retinal tissue, without affecting adjacent, healthy retinal tissue, those of ordinary skill in the art will understand the applicability of the disclosed invention for selectively treating other ocular tissues. Generally, therefore, the particular ocular tissue to which the disclosed invention is directed does not limit the invention.

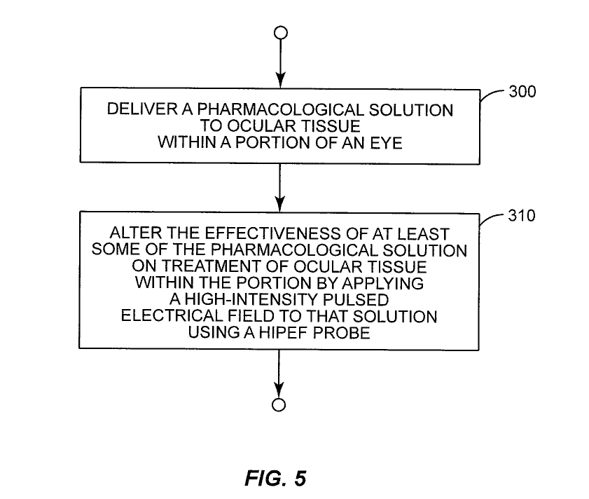

Accordingly, those of ordinary skill in the art will readily appreciate that the HIPEF apparatus 200 generally performs the method illustrated in FIG. 5 for treating ocular tissue within a localized portion of an eye 100 with a pharmacological solution. As shown in FIG. 5, the irrigation system 130 of the apparatus 200 delivers a pharmacological solution to ocular tissue within a portion of the eye (Block 100). The HIPEF probe 110 then alters the effectiveness of at least some of the pharmacological solution on treatment of ocular tissue within the portion by applying a HIPEF to that solution (Block 110).

Of course, this embodiment and all of the other embodiments described above for treating ocular tissue within a localized portion of an eye were given for purposes of illustration and example. Those skilled in the art will appreciate, therefore, that the present invention may be carried out in other ways than those specifically set forth herein without departing from essential characteristics of the invention. The present embodiments are thus to be considered in all respects as illustrative and not restrictive, and all changes coming within the meaning and equivalency range of the appended claims are intended to be embraced therein.

What is claimed is:

1. A method for treating ocular tissue within a localized portion of an eye during eye surgery with a pharmacological solution, the method comprising:
delivering a pharmacological solution to ocular tissue within a portion of an eye; and
altering the effectiveness of at least some of the pharmacological solution on treatment of ocular tissue within said portion by applying a high-intensity pulsed electrical field to said at least some solution, using a high-intensity pulsed-electrical-field (HIPEF) probe.

2. The method of claim 1, wherein delivering a pharmacological solution to ocular tissue within a portion of the eye comprises delivering a pharmacological carrier encapsulating an active pharmacological solution to said ocular tissue.

3. The method of claim 2, wherein altering the effectiveness of at least some of the pharmacological solution on treatment of ocular tissue within said portion by applying a high-intensity pulsed electrical field to said at least some solution comprises penetrating the pharmacological carrier, and thereby exposing the ocular tissue to the active pharmacological solution, by applying the high-intensity pulsed electrical field to said pharmacological carrier.

4. The method of claim 2, wherein delivering a pharmacological carrier encapsulating an active pharmacological solution comprises delivering a liposome carrier encapsulating the active pharmacological solution.

5. The method of claim 1, wherein delivering a pharmacological solution to ocular tissue within a portion of the eye comprises delivering an inactive pharmacological solution to said ocular tissue.

6. The method of claim 1, wherein altering the effectiveness of at least some of the pharmacological solution on treatment of ocular tissue within said portion by applying a high-intensity pulsed electrical field to said at least some solution comprises activating at least some of said inactive pharmacological solution by applying the high-intensity pulsed electrical field to said at least some inactive pharmacological solution.

7. The method of claim 1, wherein delivering a pharmacological solution to ocular tissue within a portion of an eye comprises delivering a highly concentrated pharmacological solution to said ocular tissue, in a low dose.

8. The method of claim 1, wherein delivering a pharmacological solution to ocular tissue within a portion of an eye comprises delivering the pharmacological solution to retinal tissue within a portion of the eye.

9. The method of claim 1, further comprising dissociating and removing vitreous tissue adjacent said ocular tissue with said HIPEF probe, and, responsive thereto, delivering the pharmacological solution to said ocular tissue and altering the effectiveness of at least some of the pharmacological solution.

10. A high-intensity pulsed-electrical-field (HIPEF) apparatus for treating ocular tissue within a localized portion of an eye during eye surgery with a pharmacological solution, the HIPEF apparatus comprising:
an irrigation system configured to deliver a pharmacological solution to ocular tissue within a portion of an eye; and
a HIPEF probe configured to alter the effectiveness of at least some of the pharmacological solution on treatment of ocular tissue within said portion by applying a high-intensity pulsed electrical field to said at least some solution.

11. The HIPEF apparatus of claim 10, wherein the irrigation system is configured to deliver a pharmacological solution to ocular tissue within a portion of the eye by delivering a pharmacological carrier encapsulating an active pharmacological solution to said ocular tissue.

12. The HIPEF apparatus of claim 11, wherein the HIPEF probe is configured to alter the effectiveness of at least some of the pharmacological solution on treatment of ocular tissue within said portion by penetrating the pharmacological carrier, and thereby exposing the ocular tissue to the active pharmacological solution, by applying the high-intensity pulsed electrical field to said pharmacological carrier.

13. The HIPEF apparatus of claim 11, wherein the HIPEF probe is configured to deliver a liposome carrier encapsulating the active pharmacological solution.

14. The HIPEF apparatus of claim 10, wherein the irrigation system is configured to deliver a pharmacological solution to ocular tissue within a portion of the eye by delivering an inactive pharmacological solution to said ocular tissue.

15. The HIPEF apparatus of claim 14, wherein the HIPEF probe is configured to alter the effectiveness of at least some of the pharmacological solution on treatment of ocular tissue within said portion by activating at least some of said inactive pharmacological solution, by applying the high-intensity pulsed electrical field to said at least some inactive pharmacological solution.

16. The HIPEF apparatus of claim 10, wherein the irrigation system is configured to deliver a pharmacological solution to ocular tissue within a portion of the eye by delivering a highly concentrated pharmacological solution to said ocular tissue, in a low dose.

17. The HIPEF apparatus of claim 10, wherein the irrigation system is configured to deliver a pharmacological solution to retinal tissue within a portion of the eye.

18. The HIPEF apparatus of claim 10, wherein the HIPEF apparatus is further configured to dissociate and remove vitreous tissue adjacent said ocular tissue, and wherein the irrigation system and HIPEF probe are configured to deliver the pharmacological solution to said ocular tissue and alter the effectiveness of at least some of the pharmacological solution responsive to said dissociation and removal of vitreous tissue.
